(19) United States
(12) Patent Application Publication
(10) Pub. No.: US 2025/0283021 A1
(43) Pub. Date: Sep. 11, 2025

(54) HUMAN CELL DERIVED MICROFLUIDIC DEVICES, SYSTEMS, AND METHODS

(71) Applicant: The University of North Carolina at Chapel Hill, Chapel Hill, NC (US)

(72) Inventors: William Joseph Polacheck, Chapel Hill, NC (US); Anthony James Hickey, Chapel Hill, NC (US); Matthew Jeffrey Rich, Chapel Hill, NC (US); Elizabeth Louise Doherty, Durham, NC (US)

(73) Assignee: The University of North Carolina at Chapel Hill, Chapel Hill, NC (US)

(21) Appl. No.: 18/858,828

(22) PCT Filed: Apr. 24, 2023
(86) PCT No.: PCT/US2023/019672

§ 371 Date: Oct. 22, 2024

Related U.S. Application Data
(60) Provisional application No. 63/333,981, filed on Apr. 22, 2022.

(51) Int. Cl. C12M 3/06; C12M 1/00; C12M 1/12

(52) U.S. Cl. CPC C12M 23/16; C12M 23/22; C12M 25/14

(57) ABSTRACT

A method for producing a microfluidic device, the method comprising: producing a first housing portion and a second housing portion; securing the second housing portion to the first housing portion; enclosing a three-dimensional biomaterial structure between the first housing portion and the second housing portion; and forming one or more channel within the biomaterial structure, the one or more channel being configured to model a hollow tissue structure; wherein the biomaterial structure and the one or more channel are configured for modeling a cellular transport barrier in a flow environment.

### CROSS REFERENCE TO RELATED APPLICATIONS

This application claims priority to and the benefit of U.S. Provisional Patent Application Ser. No. 63/333,981, filed Apr. 22, 2022; the disclosure of which is incorporated herein by reference in its entirety.

### GOVERNMENT SUPPORT

This invention was made with government support under Grant No. GM142944 awarded by the National Institutes of Health. The government has certain rights in the invention.

### TECHNICAL FIELD

The subject matter disclosed herein relates generally to devices, systems, and methods for modeling human cell interactions in vitro. More particularly, in some embodiments, the subject matter disclosed herein relates to experimental platforms for modeling a cellular transport barrier in a flow environment.

### BACKGROUND

Investigating the molecular mechanisms that regulate human vascular and other cellular barrier functions is complicated by the lack of experimental systems that enable precise control of the mechanical and chemical endothelial microenvironments. In vivo, the ability to modulate blood pressures and flows is limited, and the mechanical effects of blood flow cannot be decoupled from changes in nutrient exchange. In vitro, despite the increased development of microfluidic vascular platforms, standard commercial assays that enable investigation of endothelial cells under flow require culturing of cells on flat, stiff substrates, which influences cell-matrix and cell-cell signaling pathways known to modulate permeability. A more complete understanding of the molecular mechanisms governing barrier function requires the development of platforms that enable culture of endothelial cells in a physiologic extracellular matrix (ECM) with appropriate stromal cells and precise control over blood flow.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to a method for producing a microfluidic device. In some embodiments, the method comprises producing a first housing portion and a second housing portion; securing the second housing portion to the first housing portion; enclosing a three-dimensional biomaterial structure between the first housing portion and the second housing portion; and forming one or more channel within the biomaterial structure, the one or more channel being configured to model a hollow tissue structure; wherein the biomaterial structure and the one or more channel are configured for modeling a cellular transport barrier in a flow environment. In some embodiments, producing the first housing portion and the second housing portion comprises using a fabrication protocol selected from the group consisting of photolithography, injection molding, and embossing. In some embodiments, one or both of the first housing portion or the second housing portion comprises a substantially optically transparent material.

In some embodiments, enclosing the three-dimensional biomaterial structure between the first housing portion and the second housing portion comprises positioning the first housing portion and the second housing portion to apply fluid pressure to the biomaterial structure. In some embodiments, enclosing the three-dimensional biomaterial structure between the first housing portion and the second housing portion comprises inserting liquid biomaterial between the first housing portion and the second housing portion; and polymerizing the liquid biomaterial to form the three-dimensional biomaterial structure.

In some embodiments, the biomaterial structure comprises a hydrogel. In some embodiments, enclosing the three-dimensional biomaterial structure between the first housing portion and the second housing portion comprises positioning a lyophilized hydrogel between the first housing portion and the second housing portion; and supplying water to the lyophilized hydrogel to reconstitute the biomaterial structure.

In some embodiments, one or both of the first housing portion or the second housing portion comprises one or more alignment feature; and wherein forming each of the one or more channel comprises aligning a tubular structure with the one or more alignment feature prior to enclosing the three-dimensional biomaterial structure between the first housing portion and the second housing portion; and removing the tubular structure to create a cylindrical void that defines the one or more channel within the biomaterial structure. In some embodiments, the tubular structure comprises a needle having a diameter in a range from about 0.12 mm to about 0.35 mm. In some embodiments, the tubular structure is coated with a material configured to inhibit adhesion of the tubular structure to the biomaterial structure. In some embodiments, the tubular structure comprises a dissolvable needle. In some embodiments, the method further comprises seeding cells in the biomaterial structure.

The presently disclosed subject matter relates in some embodiments to a microfluidic device. In some embodiments, the microfluidic device comprises a first housing portion; a second housing portion secured to the first housing portion; a three-dimensional biomaterial structure enclosed between the first housing portion and the second housing portion; and one or more channel formed within the biomaterial structure;

wherein the biomaterial structure and the one or more channel are configured for modeling a cellular transport barrier in a flow environment. In some embodiments, one or both of the first housing portion or the second housing portion comprises a substantially optically transparent material. In some embodiments, the first housing portion comprises one or more alignment features configured to facilitate positioning of the one or more channel in the biomaterial structure. In some embodiments, the first housing portion and the second housing portion are configured to apply fluid pressure to the biomaterial structure.

In some embodiment, the biomaterial structure comprises a hydrogel. In some embodiments, the biomaterial structure comprises a human-cell-derived extracellular matrix. In some embodiments, the device comprises one or more media port formed in the second housing portion in communication with the one or more channel. In some embodiments, the device comprises a flow system in communication with one of the one or more media port, wherein the flow system is configured to generate fluid flow through the one or more channel. In some embodiments, the device comprises one or more extracellular matrix port formed in the second housing portion in communication with the biomaterial structure. In some embodiments, the device comprises cells seeded in the biomaterial structure.

The presently disclosed subject matter relates in some embodiments to a method for producing a microfluidic device. In some embodiments, the method comprises producing a device housing comprising one or more internal cavity enclosed therein; positioning a three-dimensional biomaterial structure within the one or more cavity in the device housing, wherein the biomaterial structure comprises a human-cell-derived extracellular matrix; and forming one or more channel within the biomaterial structure, the one or more channel being configured to model a hollow tissue structure; wherein the biomaterial structure and the one or more channel are configured for modeling a cellular transport barrier in a flow environment. In some embodiments, the device housing comprises a substantially optically transparent material.

In some embodiments, positioning the three-dimensional biomaterial structure within the one or more cavity in the device housing comprises inserting liquid biomaterial within the one or more cavity in the device housing; and polymerizing the liquid biomaterial to form the three-dimensional biomaterial structure. In some embodiments, the biomaterial structure comprises a hydrogel. In some embodiments, positioning the three-dimensional biomaterial structure within the one or more cavity in the device housing comprises positioning a lyophilized hydrogel within the one or more cavity in the device housing; and supplying water to the lyophilized hydrogel to reconstitute the biomaterial structure.

In some embodiments, forming each of the one or more channel comprises positioning a tubular structure within the one or more cavity in the device housing prior to enclosing the three-dimensional biomaterial structure within the one or more cavity in the device housing; and removing the tubular structure to create a cylindrical void that defines the one or more channel within the biomaterial structure. In some embodiments, the tubular structure comprises a needle having a diameter in a range from about 0.12 mm to about 0.35 mm. In some embodiments, the tubular structure is coated with a material configured to inhibit adhesion of the tubular structure to the biomaterial structure. In some embodiments, the tubular structure comprises a dissolvable needle. In some embodiments, the method further comprises seeding cells in the biomaterial structure.

The presently disclosed subject matter relates in some embodiments to a microfluidic device. In some embodiments, the microfluidic device comprises a device housing comprising one or more cavity enclosed therein; a three-dimensional biomaterial structure positioned within the one or more cavity of the device housing, wherein the biomaterial structure comprises a human-cell-derived extracellular matrix; and one or more channel formed within the biomaterial structure; wherein the biomaterial structure and the one or more channel are configured for modeling a cellular transport barrier in a flow environment. In some embodiments, the device housing comprises an optically transparent material.

In some embodiments, the biomaterial structure comprises a hydrogel. In some embodiments, the device comprises one or more media port formed in the device housing in communication with the one or more channel. In some embodiments, the device comprises a flow system in communication with one of the one or more media port, wherein the flow system is configured to generate fluid flow through the one or more channel. In some embodiments, the device comprises one or more extracellular matrix port formed in the second housing portion in communication with the biomaterial structure. In some embodiments, the device comprises cells seeded in the biomaterial structure.

Accordingly, it is an object of the presently disclosed subject matter to provide devices, systems, and methods for modeling human cell interactions in vitro. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and Examples. Additionally, various aspects and embodiments of the presently disclosed subject matter are described in further detail below.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIG. 1 is a perspective view, a sectional view, and a top view of a microfluidic device according to an embodiment of the presently disclosed subject matter. In some embodiments, the sectional view is a view that occurs along cross section line AA.

FIG. 2 is a plan view of a two-piece mold and sectional views of components for a microfluidic device according to an embodiment of the presently disclosed subject matter. In some embodiments, the sectional views are exploded views of components of a device of the presently disclosed subject matter along the sets of lines in the right panel.

FIGS. 3A and 3B illustrate steps in a process for making a microfluidic device according to an embodiment of the presently disclosed subject matter. In some embodiments, also included, are side views and exploded sectional views of an embodiment of the presently disclosed subject matter along the sets of lines in the bottom central panel and top central panel, respectively.

FIGS. 4A through 4C illustrate steps in an injection molding process for making a microfluidic device according to an embodiment of the presently disclosed subject matter.

FIGS. 5A through 5C illustrate steps in an injection molding process for making a microfluidic device according to an embodiment of the presently disclosed subject matter.

FIGS. 6A through 6C illustrate steps in fabricating a microfluidic device from a plurality of laser-cut sheets according to an embodiment of the presently disclosed subject matter.

FIGS. 7A through 7D illustrate steps in a “dieless” reverse injection molding process for making a microfluidic device according to an embodiment of the presently disclosed subject matter.

FIGS. 8A through 8C are images of a human engineered microvessel platform cultured under flow according to an embodiment of the presently disclosed subject matter. Arrows in 8A and 8B show DAPI stained nuclei of microvessel.

FIGS. 9A through 9D are images showing the use of a microfluidic device according to an embodiment of the presently disclosed subject matter with patient blood and plasma (9A and 9B) and different primary adult human endothelial cells for patient or disease specific vascular health measurement (9C and 9D).

FIGS. 10A through 10C are graphs and images of hydrogel analysis. FIG. 10A is a turbidity analysis. FIG. 10B is a Young's modulus analysis. FIG. 10C is set of scanning electron microscopy images.

FIG. 11 shows images of microfluidic devices patterned to contain the cell-derived matrix hydrogel.

FIGS. 12A through 12B show human umbilical vein endothelial cells within cell-derived matrix hydrogels.

FIG. 13 show blood outgrowth endothelial cells derived from patient blood drawn within the microfluidic device.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined herein, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Numerical ranges recited herein by endpoints include all numbers and fractions subsumed within that range (e.g. 1 to 5 includes, but is not limited to, 1, 1.5, 2, 2.75, 3, 3.90, 4, and 5).

The term “comprising,” which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “lyophilization” refers the process of freeze drying or cryodesiccating a material for storage. In some embodiments, lyophilization is a method of drying a material without destroying the material's physical or chemical structure. In some embodiments, a lyophilized material is ground into a powder to prepare a hydrogel.

The presently disclosed subject matter provides microfluidic devices, systems, and methods that model the blood vasculature and other hollow tissue structures including ducts and vessels. In some embodiments, an aspect of the presently disclosed subject matter is a polymer housing with alignment features to allow generation of hollow tubes in hydrogels and biomaterials. In some embodiments, the presently disclosed devices are small, requiring low cell numbers and reagent volumes, making them amenable to high-throughput manufacturing and interfacing with standard laboratory equipment.

In some embodiments, a microfluidic platform that enables culture of endothelial cells in physiologic architectures is disclosed. The presently disclosed devices, systems, and methods can address the experimental needs for an in vitro platform to model human cellular transport barriers, which can include incorporating 3D ECM and coculture with cells, such as mural cells. The devices can be connected to various pumps and fluid-handling systems to simulate blood pressure and flow. Such a human engineered microvessel can be designed to enable high-resolution confocal microscopy of cells within the devices, and cells can be harvested from the devices for standard biochemical assays.

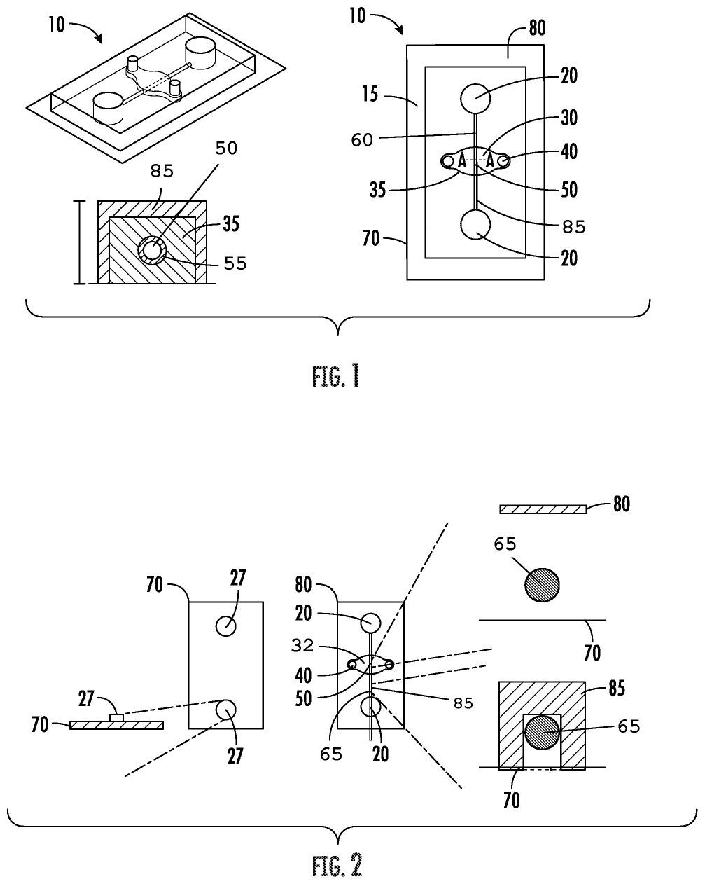

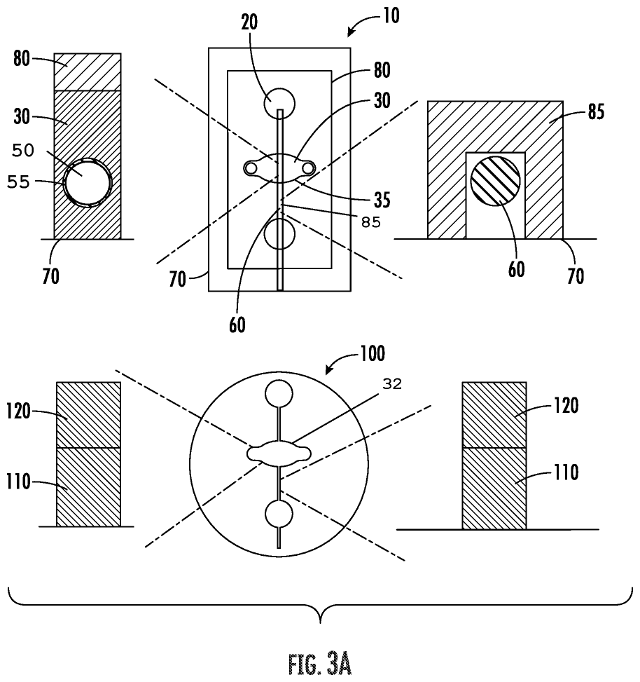

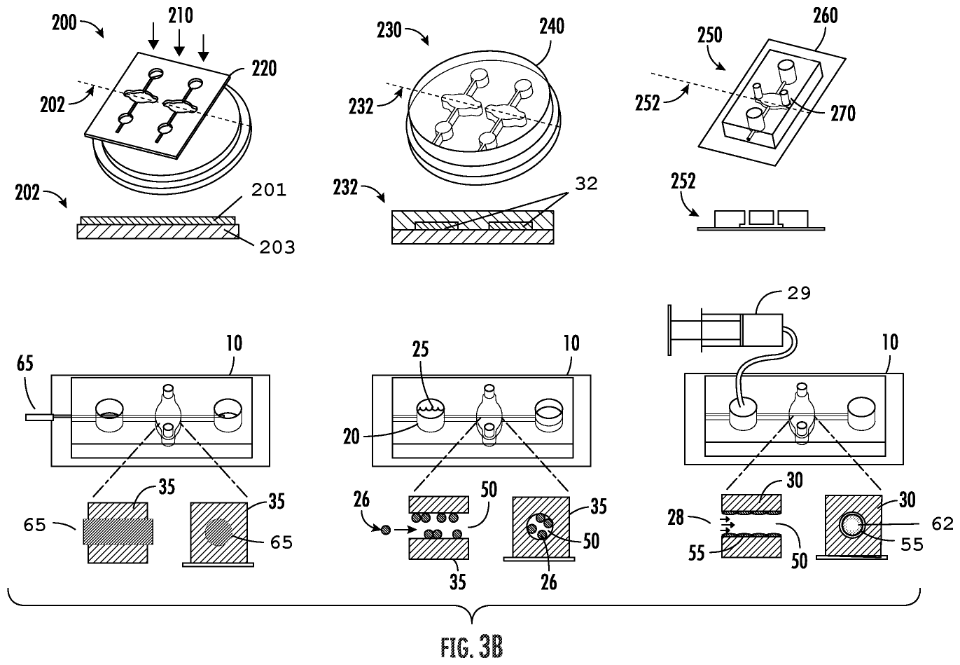

Referring now to the Figures, wherein like reference numerals refer to like parts throughout, and referring initially in particular to FIG. 1 and FIG. 2, the presently disclosed subject matter provides a microfluidic device 10 comprising a device comprising a first housing portion 70 and a second housing portion 80; a three-dimensional biomaterial structure 35 enclosed between the first housing portion 70 and the second housing portion 80; and one or more channel 60 formed within the biomaterial structure 35; wherein the biomaterial structure 35 and the one or more channel 60 are configured for modeling a cellular transport barrier 55 (FIG. 3B) at a void 50, which in some embodiments is circular or cylindrical, in a flow environment. While a cylindrical or circular void is shown, any void shape as might be apparent to one of ordinary skill in the art upon a review of the instant disclosure is provided in accordance with the instant disclosure. In some embodiments, the microfluidic device 10 comprises various dimensions and size ranges of elements. While representative, non-limiting dimensions and size ranges are provided, any suitable dimension or size range as would be apparent to one of ordinary skill in the art upon a review of the instant disclosure can be employed and are provided in accordance with the presently disclosed subject matter. Additionally, dimensions or size ranges greater than or less than those as set forth here can be employed and are provided herein. In some embodiments, the microfluidic device 10 has a length of about 50 mm or less, in some embodiments ranging from about 10 mm to about 50 mm. In an exemplary embodiment, the microfluidic device 10 has a length of about 34 mm or less. In some embodiments, the microfluidic device 10 has a height of about 10 mm or less, in some embodiments ranging from about 1 mm to about 10 mm. In an exemplary embodiment, the microfluidic device 10 has a height of about 4 mm or less.

In some embodiments, the microfluidic device 10 comprises one or more media port 20. In some embodiments, the one or more media port 20 has a diameter range of about 10 mm or less, in some embodiments about 1 mm to about 10 mm. In an exemplary embodiment, the one or more media port 20 has a diameter of about 4 mm or less. In some embodiments, the microfluidic device 10 comprises one or more extra cellular matrix (ECM) port 40. In some embodiments, the ECM port 40 has a diameter range of about 10 mm or less, in some embodiments ranging from about 1 mm to about 10 mm. In an exemplary embodiment, the ECM port 40 has a diameter of about 2 mm or less. In some embodiments, the microfluidic device 10 comprises a biomaterial 30 disposed in cavity 32. In some embodiments, the biomaterial 30 comprises a biomaterial structure 35. In some embodiments, biomaterial structure 35 comprises the cylindrical void 50 comprising barrier 55. In an exemplary embodiment, biomaterial 30 is inserted into the cavity 32 by way of the ECM port 40 to form the biomaterial structure 35.

In some embodiments, the microfluidic device 10 comprises one or more channel 60. In some embodiments, the one or more channel 60 is configured to connect one or more port 20 to the biomaterial 30 of the biomaterial structure 35. In some embodiments, at least a portion of the one or more channel 60 has a length of about 20 mm or less, in some embodiments a length range of about 1 mm to about 20 mm. In an exemplary embodiment, at least a portion of the one or more channel 60 has a length of about 5 mm or less. In some embodiments, the one or more channel 60 has a width or diameter that reflects a diameter of needle 65. In some embodiments, the one or more channel 60 has a width or diameter of about 500 μm or less, in some embodiments a width or diameter range of about 50 μm to about 500 μm. In an exemplary embodiment, the one or more channel 60 has a width or diameter of 160 μm or less. In some embodiments, the one or more channel 60 is positioned (such as by using guide 85) relative to a periphery or edge of device 10 (e.g. of first housing portion 70 and/or or second housing portion 80) at a height range of about 300 μm or less, in some embodiments a height range of about 100 μm to about 300 μm. In an exemplary embodiment, the one or more channel 60 has a height relative to a periphery or edge of device 10 (e.g. of first housing portion 70 and/or or second housing portion 80) at of 250 μm or less. In some embodiments, the height of the one or more channel is measured from a periphery or edge of the channel on a opposite side of the channel from a reference periphery or edge of the device 10.

In some embodiments, the microfluidic device 10 comprises a channel guide 85. In some embodiments, the channel guide 85 is configured to direct the one or more channel 60 (or to direct a needle 65 used in some embodiments to form channel 60) to the port 20.

In some embodiments, the channel guide 85 is configured to direct the one or more channel 60 to the biomaterial structure 35 containing the biomaterial 30, or to direct a needle 65 used in some embodiments to form channel 60. In an exemplary embodiment, the one or more channel 60 and cylindrical void 50 are formed by placing the needle 65 in the microfluidic device 10 and then filling the device with biomaterial 30. In an exemplary embodiment, once the biomaterial 30 has set to form the biomaterial structure 35, the needle 65 is removed from the microfluidic device 10 to create cylindrical void 50. In an exemplary embodiment, the needle 65 is inserted into the one or more channel 60 to punch cylindrical void 50 into biomaterial structure 35 after biomaterial 30 has been poured into cavity 32 and allowed to set.

In some embodiments, one or both of the first housing portion 70 or the second housing portion 80 comprises a substantially optically transparent material. In some embodiments, the substantially optically transparent material is described elsewhere herein. In some embodiments, the first housing portion 70 and the second housing portion 80 are configured to apply fluid pressure to the biomaterial structure 35. In some embodiments, the biomaterial structure 35 comprises a hydrogel. In some embodiments, the biomaterial structure 35 comprises a human-cell-derived extracellular matrix. In some embodiments, the biomaterial structure comprises cellular transport barrier 55.

In some embodiments, microfluidic device 10 comprises one or more media port 20 formed in the second housing portion 80 in communication with the one or more channel 60. In some embodiments, the microfluidic device 10 comprises a flow system in communication with one of the one or more media port 20, wherein the flow system 29 (FIG. 3B) is configured to generate fluid flow through the one or more channel 60.

In some embodiments, the microfluidic device 10 comprises one or more extracellular matrix port 40 formed in the second housing portion 80 in communication with the biomaterial structure one or more extracellular matrix port formed in the second housing portion in communication with the biomaterial structure 35.

Referring to FIG. 2, in some embodiments one or more molds, such as but not limited to a bottom mold and a top mold, are used in the preparation of first housing portion 70 and second housing portion 80, such as by using an injection mold/embossing process. In some embodiments, the bottom mold for first housing portion 70 and/or top mold for second housing portion 80 are each used to form one or more alignment features 27 configured to facilitate positioning of the one or more channel 60 in the biomaterial structure 35, to form ports 20 and 40 and to form cavity 32 for biomaterial 30 in which to form biomaterial structure 35. Thus, in some embodiments, microfluidic device 10 can be produced efficiently from a two-piece housing. Referring again to FIG. 2, the bottom mold can be used to define a base for the structure, e.g., first housing portion 70, and the top mold can provide second housing portion 80 so that second housing portion 80 can be secured to the first housing portion 70. The bottom and/or top mold can define one or more internal cavities 32 or one or more media port 20 or one or more matrix port 40 and provide various ports 20 or 40 that are desired (e.g., having diameters ranging from about 1.5 mm to about 5 mm depending on application, with a ‘standard’ diameter being about 5 mm for media ports 20). In some embodiments, depending on the material used to form the housing 15 of the microfluidic device 10, the housing 15 can be configured for the end-user to determine the port 20 locations and/or configurations, such as by using a biopsy punch. In some embodiments, the device 10, and/or one or more molds (e.g., bottom and/or top molds used to prepare device 10) includes a channel guide 85, which is configured in some embodiments to direct the one or more channel 60 to the cavity 32 for biomaterial structure 35 containing the biomaterial 30, or to direct a needle 65 used in some embodiments to form channel 60. In some embodiments, channel guide 85 can comprise molded plastic.

Referring now to FIG. 3A, in some embodiments, the microfluidic device 10 comprises a first housing portion 70 that supports biomaterial 30 and biomaterial 30 supports the cylindrical void 50 in which barrier 55 is found. In some embodiments, the first housing portion 70 comprises glass. In some embodiments, the microfluidic device 10 comprises a second housing portion 80 that covers biomaterial 30 and provides the channel guide 85 for one or more channel 60. In some embodiments, the second housing portion 80 comprises polydimethylsiloxane (PDMS). In some embodiments, biomaterial 30 supports the one or more channel 60. In some embodiments, the biomaterial comprises a thickness of to about 500 μm or less, in some embodiments a thickness range of about 100 to about 500 μm. In an exemplary embodiment, the biomaterial comprises a thickness of about 250 μm or less. In some embodiments, the channel guide 85 is configured to support one or more channel 60 and to contact the first housing portion 70, or is configured in some embodiments to direct the one or more channel 60 to the cavity 32 for biomaterial structure 35 containing the biomaterial 30, or to direct a needle 65 used in some embodiments to form channel 60.

Continuing with FIG. 3A, the microfluidic device can be configured as a silicon wafer device 100. In some embodiments, the silicon waiver device 100 is configured to support a first layer 110 and a second layer 120 of biomaterial structure. In some embodiments, the silicon waiver device 100 is configured to support the one or more channel within the first layer 110 and the second layer 120. In some embodiments, the first layer 110 and the second layer 120 of biomaterial structure comprise a thickness of about 500 μm or less, including a thickness range of about 100 to about 500 μm each. In an exemplary embodiment, the first layer 110 and the second layer 120 of biomaterial structure comprise a thickness of 250 μm or less each.

Referring now to FIG. 3B, in some embodiments, the microfluidic device comprises a photolithography microfluidic device 200. In some embodiments, device 200 comprises a photoresist material layer 201 and silicon layer 203. In some embodiments, the photolithography microfluidic device 200 comprises a transparency mask 220 exposed to ultraviolet radiation 210. In some embodiments, the photolithography microfluidic device 200 is shown along sectional viewpoint 202.

Continuing with FIG. 3B, in some embodiments, the microfluidic device comprises a soft lithography microfluidic device 230. In some embodiments, the soft lithography microfluidic device 230 is prepared by pouring PDMS on a mold template 240. In some embodiments, the poured PDMS cures on the mold template 240. In some embodiments, the soft lithography microfluidic device 230 is shown along sectional viewpoint 232 in which cavities 32 are visible. Continuing with FIG. 3B, in some embodiments, the microfluidic device comprises a chip preparation 250. In some embodiments, the chip preparation 250 comprises a chip, wherein the chip is cut, bonded and/or surface-treated. In some embodiments, the chip is configured to be punched and maintain a punched structure 270. In some embodiments, the chip 250 is shown along sectional viewpoint 252.

Continuing with FIG. 3B, in some embodiments, the microfluidic device 10 comprises a needle 65 that inserts into the one or more channel 60 residing in the biomaterial structure 35. In some embodiments, a cell suspension 25 is introduced to one or more media port 20 wherein cells 26 of the cell suspension 25 flow through cylindrical void 50 residing in the biomaterial structure 35 and comprising barrier 55. Cells 26 can form a tissue structure, such as a hollow tissue structure, such as a vessel 62 for study, as described herein. In some embodiments, a fluid flow device 29 is configured to provide a fluid flow 28 through cylindrical void 50 residing in the biomaterial structure 35. In some embodiments, the fluid flow device comprises a pump. In some embodiments, the pump comprises a positive displacement pump. In some embodiments, the pump comprises a centrifugal pump. In some embodiments, the pump comprises an axial-flow pump. In some embodiments, the pump comprises a paper pump. In some embodiments, the pump comprises an osmotic pump. In some embodiments, the pump is selected from the group comprising a positive displacement pump, a syringe pump, an impulse pump, a velocity pump, a gravity pump, a stream pump, a valveless pump and any combination thereof.

In one aspect, the presently disclosed subject matter provides methods and systems for producing a microfluidic device 10 that involves casting a three-dimensional biomaterial structure around a needle 65 (see FIG. 3B) (e.g., a stainless-steel needle) or other elongated, substantially tubular structure. When the biomaterial structure 30 is sufficiently solidified, the needle 65 can be removed to create a cylindrical void 50 in the structure that serves as a template for seeding cells and the eventual vessel lumen. The biomaterial structure 30 is confined within a microfluidic device 10 that includes ports 20 to access the vessel lumen and serves as a connection point for fluid flow devices (e.g., pumps) 29 (see FIG. 3B) or other fluid-handling systems. An example configuration of such a microfluidic device 10 is shown in FIG. 1.

The efficiency of construction of the two-part housing allows for it to be fabricated using any of a variety of rapid fabrication protocols, including but not limited to photolithography, injection molding (e.g., monolithic or multipart), hot embossing, replica molding of patterned substrates, or additive manufacturing. In some embodiments, it is desirable that one or both of the housing portions be composed of an optically transparent material. In particular, in some embodiments, higher-resolution imaging can be more readily achieved using plastics having indices of refraction that match or are close to that of glass. Depending on the fabrication technique used, suitable materials include but are not limited to glass, polydimethylsiloxane (PDMS), polydimethyl siloxane, polystyrene, polycarbonate, polytetrafluoroethylene (PTFE), perfluoroalkoxy (PFA), fluorinated ethylene-polypropylene (FEP), or cyclic olefin copolymer (COC). In addition, in some embodiments, the material can be sufficiently rigid to be handled for needle introduction and removal, it is possible to sterilize the material, and the material should be amenable to surface modification for covalent attachment of biomaterials and/or hydrogels. Further, in some embodiments, it is desirable that the material exhibit low auto-fluorescence. Using any of these protocols or materials, the housing can be constructed in a manner that is simpler and more repeatable than conventional methods, reducing fabrication time and more easily enabling batch production.

Referring to FIGS. 3A and 3B, in some embodiments, fabrication of the device can involve forming a mold of the internal structure of the device, such as by using photolithography 200. The two-part housing can be constructed using this mold, such as by injection molding or embossing, which allows the use of hard plastics as discussed above. With this construction, only a single alignment step is necessary at the construction stage, and thus a wafer 220 containing the microfluidic device can be constructed in about 30 to about 60 minutes. As further shown in FIG. 3B, the device 250 can then be cut forming a top plate 270 and bonded to a substrate 260 (e.g., glass, polydimethylsiloxane (PDMS), polydimethyl siloxane, polystyrene, polycarbonate, polytetrafluoroethylene (PTFE), perfluoroalkoxy (PFA), fluorinated ethylene-polypropylene (FEP) and/or cyclic olefin copolymer (COC).

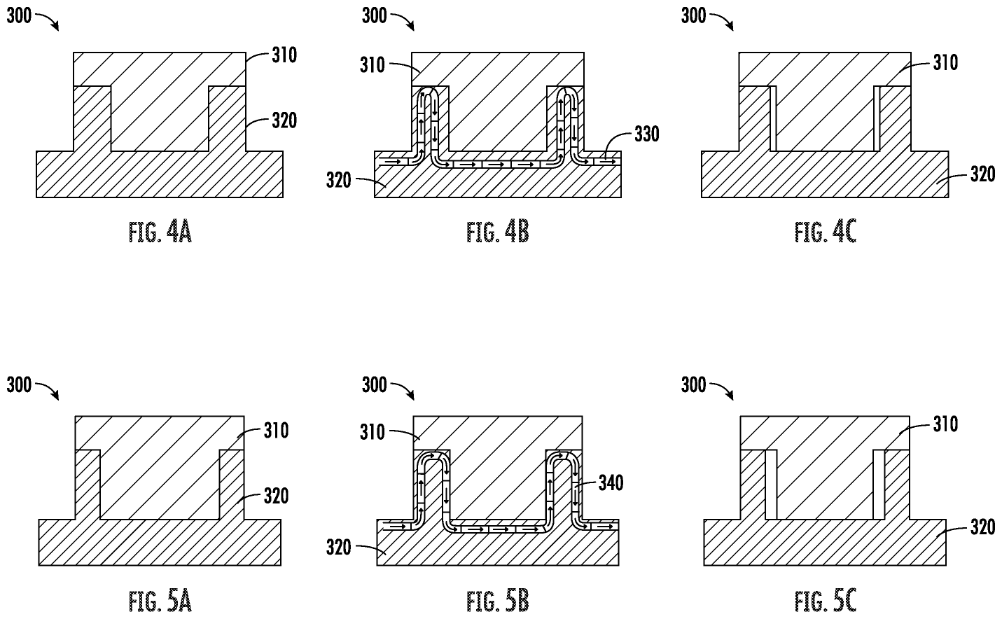

Referring to FIGS. 4A through 5C, in some embodiments, a zero draft angle mold release mechanism 300 can be used in the housing fabrication. In a first process, mold dimensions can be designed to match the specifications of the desired part 310 (FIG. 4A), an injection molding process can then implemented (FIG. 4B), and active cooling 330 can be applied to the mold 320 to shrink the mold 320 away from the part 310 to allow for release (FIG. 4C). Alternatively in a second process, mold dimensions can be designed to exceed the specification of the desired part (FIG. 5A), the mold 320 can be actively heated 340 during an injection molding process to thermally expand features to desired specifications (FIG. 5B), and the mold 320 will then be allowed to shrink away from part 310 as the assembly passively cools, allowing for part release (FIG. 5C).

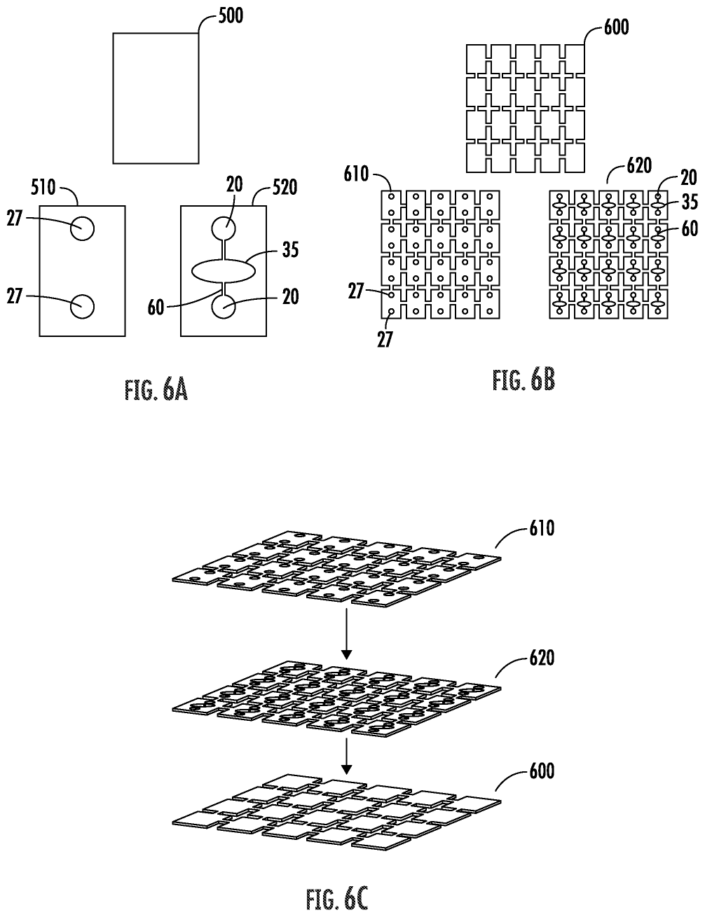

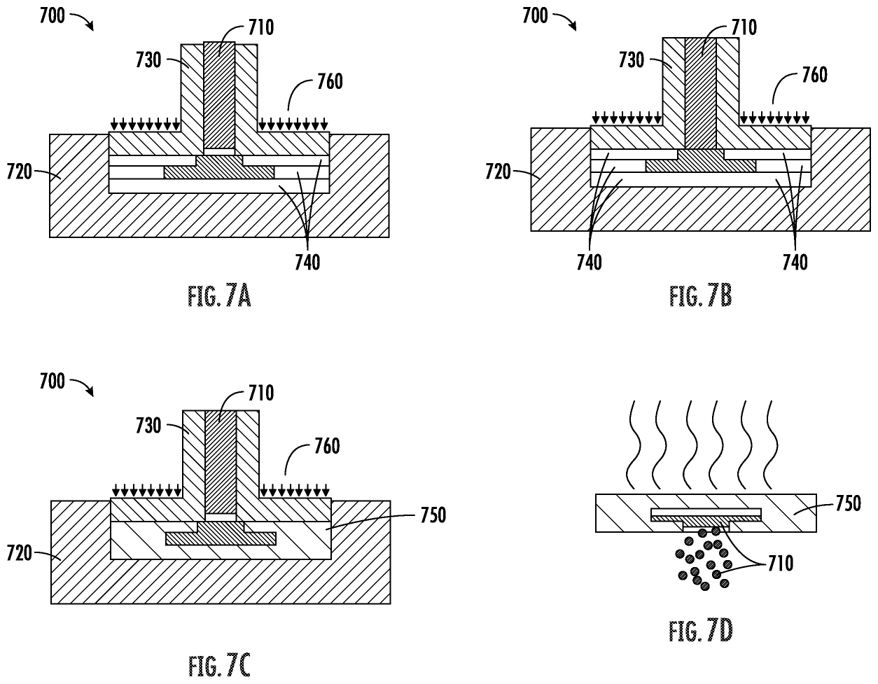

Referring to FIGS. 6A through 7D, in some embodiments, the housing can be fabricated using a “dieless” reverse injection molding process. The housing design can be modified for batch processing by deconstructing into axial layers, such as is shown in

FIGS. 6A and 6B. The patterns (500, 510, 520, 600, 610 & 620) can then be aligned (See, e.g., FIG. 6C) before insertion into an injection mold. Referring to FIG. 7A, the injection mold mechanism 700 creates a hermetic seal of the negative space in the layers 740 of the microfluidic chip. Referring to FIG. 7B, heat 760 is applied to a thermally reversible polymer allowing for the flow of support material 710 into the chip. Then, as shown in FIG. 7C, heat 760 is applied to the mold to fuse the separate layers 740 into one continuous piece 750. Finally, as shown in FIG. 7D, the device(s) is removed from the mold 720, and the support material 710 can be dissolved out of the microfluidic device. This configuration thus effectively molds the negative space, eliminating the need for costly die fabrication. In addition, this process can be used for injection molding components with internal cavities and undercuts not possible under traditional means.

Regardless of the particular construction of the housing, a three-dimensional biomaterial structure can be enclosed between the first housing portion 70 and the second housing portion 80. In some embodiments, the biomaterial 30 can be introduced into the housing by way of one or more ECM port 40 provided in the housing 15 (e.g., having a diameter of about 1.5 mm). One or more channel 60 can be formed within the biomaterial structure 35. In this configuration, the biomaterial structure 35 and the one or more channel 60 can be configured for modeling a desired cellular transport barrier in a flow environment.

As used herein, the term “cellular transport barrier” (in some embodiments, labeled at 55 in the Figures) should be understood to refer to a selectively permeable structure that separates the fluid contents of a vessel or duct lumen from surrounding tissue. In the case of vessels, the lumen contains blood (blood vessels) or lymph (lymphatic vessels), while the contents of ducts varies significantly with tissue. For example, mammary ducts contain milk while bile ducts contain bile in the luminal space. The inner lining of vessels and ducts, which is in contact with the fluid in the lumen, is formed by cells (endothelial cells for vessels and epithelial cells for ducts), and these cells selectively restrict or allow cells and molecules to pass from tissue to the lumen or vice versa. In this regard, in some embodiments, to more effectively model the desired environments, enclosing the three-dimensional biomaterial structure between the first housing portion and the second housing portion can involve structuring and/or positioning the first housing portion and the second housing portion to apply a desired fluid pressure to the biomaterial structure, such as to model interstitial pressure surrounding a tissue and/or luminal pressure within the channels.

For this purpose, any of a variety of biomaterial structures can be used for the microfluidic device. In some embodiments, for example, the biomaterial structures can include a hydrogel. Examples of suitable hydrogel materials include but are not limited to collagen type I derived from rat tail, fibrin, Matrigel, alginate, cell-derived matrix, decellularized explant tissue, or synthetic polymers such as polyethylene glycol (PEG) or dextran. Further in some embodiments, hydrogel substrates can be surface coated with basement membrane components (e.g., fibronectin, collagen IV, or laminin).

Alternatively, in some embodiments, the biomaterial structure comprises a human-cell-derived extracellular matrix (e.g., a cell-derived matrix, a decellularized explant tissue, or fibrin). In some embodiments, the human cell-derived matrix is formed by using cells to grow matrix directly in the device. In some embodiments, the cells are seeded in the extracellular matrix (FIGS. 12A and 12B). In some embodiments, the cells comprise endothelial cells. However, any desired cell as would be apparent to one of ordinary skill in the art upon a review of the instant disclosure can be employed. In some embodiments, the cells are combined with a hollow tube configuration to build more complex tissues. Alternatively, in some embodiments, the matrix is grown separately, and then the matrix is digested in a manner that allows it to be injected into the device and polymerized as a hydrogel at a later time point. In either configuration, cells can be grown in an environment that is more similar to a natural environment than prior in vitro platforms.

In some embodiments, the presently disclosed subject matter provides a human-cell-derived extracellular matrix composition, and/or hydrogel comprising the same, wherein the animal and synthetic materials that the cells attach to are replaced, thereby providing a 3D microfluidic tissue model made from all human components (matrix & cells). In some embodiments, patient plasma can be used. Thus, in some embodiments, patient-specific devices are provided, wherein all components come from a single donor. Representative, non-limiting protocols for preparing cell-derived matrix and extracellular matrix are provided elsewhere herein. While particular examples of reagents are disclosed in the protocols, e.g., particular buffers, detergents, enzymes, starting materials, and the like, it will be appreciated by one of ordinary skill in the art that other reagents can be employed. Indeed, a variety of particular examples of steps, conditions, and other parameters are described in the protocols, but any suitable steps, conditions, or other parameters as would be apparent to one of ordinary skill in the art upon a review of the instant disclosure are provided in accordance with the presently disclosed subject matter.

With some of these compositions, enclosing the three-dimensional biomaterial structure 35 between the first housing portion 70 and the second housing portion 80 can involve inserting biomaterial 30 between the first housing portion 70 and the second housing portion 80 and polymerizing the biomaterial 30 to form the three-dimensional biomaterial structure 35 comprising a barrier 55. In some embodiments, biomaterial 30 comprises a liquid biomaterial. In some embodiments, biomaterial 30 comprises a gel biomaterial. Alternatively, in some embodiments, the biomaterial can be provided as a lyophilized hydrogel within the housing, and the end user need only add water. For example, some hydrogels, including collagen type I and fibrin, can be lyophilized to preserve mass that can be reconstituted by end-user. In addition, in some embodiments, further additives (e.g., salt and/or crosslinking agents) can be provided to modulate the mechanical properties of the biomaterial.

As discussed above, the one or more channel 60 formed within the biomaterial structure 35 can be formed by positioning a needle 65 or other elongated, substantially tubular structure within the housing 15 about which the biomaterial structure 35 and/or barrier 55 can be formed. Any of a variety of tubular structures can be used for this purpose, including but not limited to a stainless steel needle or needle-shaped structures formed of PDMS or PTFE. In some embodiments, the needle can be coated with a material configured to inhibit adhesion to the biomaterial structure. For example, suitable coatings include but are not limited to bovine serum albumin, gelatin, or fluorinated epoxy resin.

In addition, the needle can be provided in any of a range of diameters depending on the desired channel size to be modeled. In some embodiments, for example, a needle having a diameter in a range from about 0.12 mm to about 0.35 mm can provide a good balance between accurately modeling microvasculature. Those having ordinary skill in the art will recognize that smaller diameters may be more reflective of microvasculature dimensions, but needles of such size can be more difficult to use and present throughput and reproducibility challenges, whereas larger diameters can be easier to use and more reproducible, but they are less physiologic and can require increased reagents.

Regardless of the particular configuration of the tubular structure (e.g., needle) that is used to form the one or more channel within the biomaterial structure, improved manufacturability of the microfluidic device is achieved where the position of the needle can be precisely oriented relative to the housing. Referring again to the embodiments shown in FIG. 2, the two-piece construction of the housing can provide for a channel guide 85 to be integrated in molds (e.g., top and/or bottom molds) for the housing and thereby within the housing portions themselves. In such configurations, one or both of the first housing portion 70 or the second housing portion 80 comprises one or more alignment feature 27 to assist the placement of the needle.

In some embodiments with such structures, forming each of the one or more channel involves simply aligning a tubular structure with the one or more alignment feature prior to enclosing the three-dimensional biomaterial structure between the first housing portion and the second housing portion. Then, once the biomaterial structure is solidified, the tubular structure can be removed to create a cylindrical void that defines the one or more channel within the biomaterial structure. Alternatively, in some embodiments, the needle can be composed of a material that is dissolvable once positioned. In some embodiments, for example, a dissolvable needle can be composed of sugar or gelatin.

Once the microfluidic device is assembled, those having ordinary skill in the art will recognize that any of a variety of further components can be connected to or associated with the device. In some embodiments, one or more flow systems can be connected, either as a direct connection or via barbed or tapered plug, and/or one or more hydrostatic reservoirs can be attached. In some embodiments, these connections can include patterned threading for twist connect and/or luer-lock compatibility. Such fluid systems can further be connected to one or more fluid reservoirs, either via a direct connection or via tubing. In some embodiments, such reservoirs can serve as a bubble trap in addition to generating pressure. In some embodiments, the reservoirs can interface with syringe pumps or other pumping systems, or they can be filled/refilled manually.

Further, the presently disclosed microfluidic devices can be used in cooperation with a variety of common laboratory equipment. In some embodiments, for example, the device can be used with a confocal microscope (e.g., to provide improved imaging resolution), an inverted microscope, fluorescence microscopy (e.g., to enable improved measurements of vascular permeability), cell culture equipment, and/or signal generator/basic electronics (e.g., for potential readouts). In addition, in some embodiments, the present microfluidic device has the potential to interface with electrical resistance measurements for vascular health (e.g., TEER-trans endothelial/epithelial electric resistance).

These and a variety of additional methods of use are enabled through use of the presently-disclosed microfluidic devices, systems, and methods, including a variety of protocols/assays that include but are not limited to vessel and duct permeability, solute transport (e.g., spatiotemporal characterization of drug/protein transport within and across tissue barriers), cell adhesion (e.g., immune cells, tumor cells, platelets), extravasation and immune cell trafficking (e.g., trans-endothelial and trans-epithelial migration), thrombosis and hemostasis, angiogenesis, vasculogenesis, fixed end-point imaging (e.g., immunofluorescence, immunohistochemistry, histology), or spatiotemporal characterization of protein activity and localization (e.g., tracking fluorescence reporters & hybrid proteins). Further, as discussed above, the present devices can be used to observe and compare flow vs. static conditions, such as by interfacing with a variety of flow systems including commercial pumps (peristaltic, syringe, etc.), or a laboratory rocker can be used for higher throughput.

Through these and other methods, the present devices and systems can be used for a variety of data analysis purposed, including but not limited to computational image processing, immunofluorescence, immunohistochemistry, activity and concentration assays (e.g., ELISA), protein expression (e.g., western blot, immunoprecipitation, mass spec.), gene expression (e.g., PCR, RNA seq.), or mechanical testing (e.g., compliance, hydraulic conductivity/porous media characterization). The present devices and methods can further be used to study various diseases, including but not limited to vascular health, COVID-19 severity, chronic kidney disease, or other diseases in which microvasculature plays a role (e.g., diabetes, sepsis, liver fibrosis, biliary atresia, solid cancers, reperfusion injury, renal fibrosis). Also, the present devices and methods can further be used in studies for drug development (e.g., drug screening, pharmacokinetics (PK)/pharmacodynamics (PD), identify/screen patients for clinical trials).

In some embodiments, the presently disclosed subject matter provides a method for producing a microfluidic device. In some embodiments, the method comprises producing a first housing portion and a second housing portion; securing the second housing portion to the first housing portion; enclosing a three-dimensional biomaterial structure between the first housing portion and the second housing portion; and forming one or more channel within the biomaterial structure. In some embodiments, the one or more channel is configured to model a hollow tissue structure. In some embodiments, the biomaterial structure and the one or more channel are configured for modeling a cellular transport barrier in a flow environment.

In some embodiments, the method comprises producing the first housing portion and the second housing portion comprises using a fabrication protocol selected from the group consisting of photolithography, injection molding, and embossing. In some embodiments, the method comprises producing the first housing portion and the second housing portion comprises photolithography. In some embodiments, the method comprises producing the first housing portion and the second housing portion comprises injection molding. In some embodiments, the method comprises producing the first housing portion and the second housing portion comprises embossing. In some embodiments, the method comprises producing the first housing portion and the second housing portion comprises using a fabrication protocol selected from the group consisting of photolithography, injection molding, embossing, and any combination thereof.

In some embodiments of the method, one or both of the first housing portion or the second housing portion comprises a substantially optically transparent material. In some embodiments, the substantially optically transparent material comprises the substantially optically transparent material selected from the group consisting of glass, polydimethylsiloxane (PDMS), polydimethyl siloxane, polystyrene, polycarbonate, polytetrafluoroethylene (PTFE), perfluoroalkoxy (PFA), fluorinated ethylene-polypropylene (FEP), cyclic olefin copolymer (COC) and any combination thereof.

In some embodiments of the method, enclosing the three-dimensional biomaterial structure between the first housing portion and the second housing portion comprises positioning the first housing portion and the second housing portion to apply fluid pressure to the biomaterial structure.

In some embodiments of the method, enclosing the three-dimensional biomaterial structure between the first housing portion and the second housing portion comprises inserting liquid biomaterial between the first housing portion and the second housing portion; and polymerizing the liquid biomaterial to form the three-dimensional biomaterial structure. In some embodiments of the method, enclosing the three-dimensional biomaterial structure between the first housing portion and the second housing portion further comprises inserting liquid biomaterial between the first housing portion and the second housing portion; and polymerizing the liquid biomaterial to form the three-dimensional biomaterial structure. In some embodiments of the method, the biomaterial or liquid biomaterial comprises an extracellular matrix. In some embodiments of the method, the extra cellular matrix comprises a cell-derived matrix. In some embodiments of the method, the extracellular matrix comprises a decellularized explant tissue. In some embodiments of the method, the extracellular matrix comprises fibrin. In some embodiments of the method, the extracellular matrix comprises human cell-derived extracellular matrix. In some embodiments of the method, the extracellular matrix is as described above and in the Examples below.

In some embodiments of the method, the biomaterial structure comprises a hydrogel. In some embodiments of the method, the liquid biomaterial comprises polymerized hydrogel.

In some embodiments of the method, enclosing the three-dimensional biomaterial structure between the first housing portion and the second housing portion comprises positioning a lyophilized hydrogel between the first housing portion and the second housing portion; and supplying a hydrating agent to the lyophilized hydrogel to reconstitute the biomaterial structure. In some embodiments of the method, the hydrating agent comprises water. In some embodiments of the method, the hydrating agent comprises mannitol. In some embodiments of the method, the hydrating agent comprises glycine.

In some embodiments of the method, one or both of the first housing portion or the second housing portion comprises one or more alignment feature. In some embodiments, forming each of the one or more channel comprises aligning a tubular structure with the one or more alignment feature prior to enclosing the three-dimensional biomaterial structure between the first housing portion and the second housing portion; and removing the tubular structure to create a cylindrical void that defines the one or more channel within the biomaterial structure.

In some embodiments of the method, the tubular structure comprises a needle having a diameter in a range from about 0.12 mm to about 0.35 mm. In some embodiments of the method, the needle comprises an outer diameter in a range from about 0.159 mm to about 3.5 mm. In some embodiments of the method, the needle comprises an inner diameter in a range from about 0.051 mm to about 2.693 mm.

In some embodiments of the method, the tubular structure is coated with a material configured to inhibit adhesion of the tubular structure to the biomaterial structure. In some embodiments of the method, the material comprises bovine serum albumin. In some embodiments of the method, the material comprises gelatin. In some embodiments of the method, the material comprises fluorinated epoxy resin.

In some embodiments of the method, the tubular structure comprises a dissolvable needle. In some embodiments of the method, the dissolvable needle comprises a sugar. In some embodiments of the method, the dissolvable needle comprises a gelatin.

In some embodiments, the presently disclosed subject matter provides a method for producing a microfluidic device. In some embodiments, the method comprises producing a device housing comprising one or more internal cavity enclosed therein; positioning a three-dimensional biomaterial structure within the one or more cavity in the device housing, wherein the biomaterial structure comprises a human-cell-derived extracellular matrix; and forming one or more channel within the biomaterial structure. In some embodiments, the one or more channel is configured to model a hollow tissue structure. In some embodiments, the biomaterial structure and the one or more channel are configured for modeling a cellular transport barrier in a flow environment.

In some embodiments of the method, the wherein the device housing comprises a substantially optically transparent material. In some embodiments of the method, the substantially optically transparent material is as described elsewhere herein and as above.

In some embodiments of the method, positioning the three-dimensional biomaterial structure within the one or more cavity in the device housing comprises inserting liquid biomaterial within the one or more cavity in the device housing; and polymerizing the liquid biomaterial to form the three-dimensional biomaterial structure. In some embodiments of the method, the biomaterial structure is as described elsewhere herein and above. In some embodiments of the method, the biomaterial structure comprises a hydrogel.

In some embodiments of the method, positioning the three-dimensional biomaterial structure within the one or more cavity in the device housing comprises positioning a lyophilized hydrogel within the one or more cavity in the device housing; and supplying hydrating agent to the lyophilized hydrogel to reconstitute the biomaterial structure. In some embodiments, the hydrating agent is described elsewhere herein and as above. In some embodiments, the hydrating agent comprises water.

In some embodiments of the method, forming each of the one or more channel comprises positioning a tubular structure within the one or more cavity in the device housing prior to enclosing the three-dimensional biomaterial structure within the one or more cavity in the device housing; and removing the tubular structure to create a cylindrical void that defines the one or more channel within the biomaterial structure.

In some embodiments of the method, the tubular structure comprises a needle described herein and above. In some embodiments of the method, the needle comprises a diameter in a range from about 0.12 mm to about 0.35 mm.

In some embodiments of the method, the tubular structure is coated with a material configured to inhibit adhesion of the tubular structure to the biomaterial structure.

In some embodiments of the method, the tubular structure comprises a dissolvable needle as described herein and above.

In some embodiments, the presently disclosed subject matter provides a microfluidic device. In some embodiments, the microfluidic device comprises a device housing comprising one or more cavity enclosed therein; a three-dimensional biomaterial structure positioned within the one or more cavity of the device housing, wherein the biomaterial structure comprises a human-cell-derived extracellular matrix; and one or more channel formed within the biomaterial structure; wherein the biomaterial structure and the one or more channel are configured for modeling a cellular transport barrier in a flow environment.

In some embodiments, the device housing comprises a substantially optically transparent material as described elsewhere herein and above.

In some embodiments, the biomaterial structure comprises a hydrogel as described elsewhere herein and above.

In some embodiments, the microfluidic device comprises one or more media port formed in the device housing in communication with the one or more channel as described herein and above.

In some embodiments, the microfluidic device comprises a flow system in communication with one of the one or more media port, wherein the flow system is configured to generate fluid flow through the one or more channel.

In some embodiments, the microfluidic device comprises one or more extracellular matrix port formed in the second housing portion in communication with the biomaterial structure.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### dECM Hydrogel Full Protocol

### Cell-Derived Matrix (CDM) Protocol

Use the deeper 10-cm dish to prevent spilling on the rocker.
For all washes, use 5 mL of reagent per plate per wash.

### Reagents for Preparing Plates, Plating CDM, and Growing CDM:

0.2% Gelatin
Phosphate buffered solution (PBS)
1 M Glycine
Growth Medium: Dulbecco's Modified Eagle Medium (DMEM)+1 g/L D-Glucose+L-Glutamine+110 mg/mL Sodium Pyruvate+10% FBS (Gibco Ref#:11885-084)
L-ascorbic acid (BioXtra, ≥99.0%, crystalline) (Sigma Ref#:A5960-25G)
   D0 (or before)|Prepare Culture Dishes for Culturing Cells to Produce CDM.
1. Wash plate 1× w/PBS in TC Hood
2. Add 5 mL of 0.2% Gelatin to each dish and incubate at 37 C. for 1 hour
   a. Gelatin stock is 2%, therefore, use C₁V₁=C₂V₂ to determine volume of 1× PBS and 2% Gelatin needed to get desired volume of 0.2% Gelatin.
3. Wash 3× with PBS
4. Add 5 mL of growth medium to each plate and incubate at 37 C. for 30 min.
5. Wash 3× with PBS
6. Use Immediately or Store at 4 C. in PBS
   a. If you store, you should incubate with growth medium again for 30 min before plating cells.

### D0|Plate Cells on Dishes to Produce CDM (Human Dermal Fibroblasts (HDF)) Passage HDFs as Normal and Resuspend to 1×10⁶ Cells/mL.

1. Add 8 mL growth media to each plate
2. Add 1.48 mL of cell solution to each plate (10-cm dishes)
   a. This should plate cells at confluency. If cells are not confluent, wait until they are confluent and then move on to growing CDM section.
   b. Proceed to next step after 24 hrs.
      D1-D6|Grow CDM from Cells Plated on Dishes.
1. Change media and supplement with 50 μg/mL ascorbic acid daily.
   a. Make a solution of ascorbic acid at 50 mg/mL
      i. Measure ascorbic acid to be between 30 mg and 50 mg (Mmeasured) and add to minicentrifuge tube.
      ii. Use the equation V [mL]=M_{measured}/50 mg to determine amount of DI H₂O to add to the amount of ascorbic acid measured.
      iii. In the biosafety cabinet add V mL of DI H₂O to measured ascorbic acid. Mix until solution is clear. Sterile filter using 0.2 μm pore size syringe filters into new microcentrifuge tube using 1 mL syringe. Use within 24 hrs.
   b. Add ascorbic acid to media to a final concentration of 50 μg/mL (1000×) before adding to the cells to replace media.

### Reagents for Decellularization

Labrepco BR2000 laboratory rocker in 37° C. incubator PBS
PBS++
1M NH₄OH
Triton X-100
Growth Medium: DMEM+1 g/L D-Glucose+L-Glutamine+110 mg/mL Sodium Pyruvate+10% FBS (Gibco Ref#:11885-084)
DNase I (sigma D4527-10KU)
Pen/Strep
Fungizone
   CDM Extraction Buffer (Warmed)
      mM NH₄OH+0.5% Triton X-100 in PBS
         Add 2 mL Triton X-100 to 40 mL PBS (scale up volumes if more is needed for number of plates).
         Add 2 mL 1M NH₄OH to the Triton X-100 solution
            D7 |Remove Cells from Deposited CDM (i.e., Decellularization)

Cells on the tissue culture plate are distributed throughout the cell-derived matrix. Cells are then removed during the decellularization process resulting in cell-free CDM.

### Lyse Cells Using Extraction Buffer

a. Aspirate cell culture medium from plate
b. Wash 1× PBS
c. Add extraction buffer to each plate
d. Agitate for 15 min total @37° C. Change extraction buffer every 5 min, and keep plates on the rocker for constant agitation
   Wash Extraction Buffer and Lysed Cells from Deposited CDM
e. Remove buffer gently using pipette and wash with media 3×, keeping the plate on the rocker for 5 min during each wash
f. Wash 1× with PBS++ using the rocker for 5 min
   DNase Treatment to Remove DNA from Deposited CDM
g. Add DNase I solution (2 μg/mL) (7 mL per dish)
   i. Add 1 aliquot (24 μL) to 12 mL of PBS++ (warmed)
   h. Incubate, 45 min at 37° C., on laboratory rocker
   i. Wash 3× in PBS with 5 min incubation on laboratory rocker for each wash

### Storage

j. Add Storage Solution and wrap with parafilm
   i. Storage Solution:
      1. PBS +Pen/Strep (100×)+0.25 μg/mL Fungizone (1000×)
      2. 50 mL: 49.5 mL PBS+500 μL Pen/Strep+50 μL Fungizone
   k. Store at 4 C. for <1 month.
      Lyophilization for Long-Term Storage and/or for Preparation of CDM Hydrogels—Sample Preparation
1. Ensure that the freeze dryer (Labconco FreeZone 2.5 L-50° C. benchtop freeze dryer) has reached operating temperature and pressure before preparing the samples.
2. Remove the storage solution from the CDM plates gently using a pipette.
3. Wash 3× with DI-H₂O to remove any residual storage solution.
4. Remove all DI-H₂O from last wash.
   a. For the following liquid-nitrogen freezing step, the least amount of liquid remaining is desired. If this step is omitted, filter the CDM from the water after scraping, although this can potentially cause some CDM to be lost through the filter.
5. Scrape CDM from the bottom of the dish using a cell scraper.
6. Add liquid nitrogen to the stainless-steel reservoir of a stainless-steel mortar.
7. Pipette the CDM into the stainless-steel mortar ladle and spread as thinly as possible. Place ladle on top of liquid nitrogen reservoir to freeze CDM.
8. Use pestle to break up the frozen CDM by lightly tapping it. Continue until a powder is formed.
9. Scrape the frozen CDM powder into a tube using a spatula/scraper. Move tube to the lyophilizer immediately to prevent the CDM powder from melting.

Alternatively, follow steps 1-5, transfer the CDM to a tube, and freeze at −80 C. for an hour before adding to the lyophilizer. The resulting sample still needs to powderized after lyophilization.

### Connecting Sample to Freeze Dryer

1. Cover the tube of CDM (uncapped) with a Kimwipe™ wipe and wrap with parafilm to hold in Kimwipe™ wipe in place.
2. Place the tube upright in a manufacturer-provided glass container. Add the cap to the glass container (the cap is black with a stainless-steel spout).
3. Choose which port to use and insert the stainless-steel spout into the port.
   a. This can be challenging. It may be easier to insert the cap alone first and then attach the glass container the cap after it is in place.
4. Turn the port from closed to opened. Wait for the vacuum to return to operating pressure.
5. Run the lyophilization sequence for 24 hours.
6. After 24 hrs., samples can be removed from the freeze dryer, aliquoted, and stored indefinitely.
   a. In subsequent steps, this freeze-dried, powdered cell-derived matrix is referred to as “CDM.”

### Example 2

### dECM Hydrogel

This is an exemplary process by which the CDM (from above) is converted into a hydrogel to use within microfluidic devices or more broadly as a cell culture substrate as described below.

### Reagents for Solubilization

Lyophilized, powdered CDM
Pepsin (3,200-4,500 units/mg, Sigma Aldrich P6887)
0.01 M HCl
Glass vial
   Given the small volume of our digests, a 2 mL glass vial worked well (example: Sigma Aldrich 29057-U).
Stir bar.
   The size of the stir bar relative to the glass vial can play a role. The stir bar needs to be just smaller than the diameter of the glass vial to grab the CDM on the edges as it stirs. Otherwise, clumps of CDM will get stuck in the outer edges.

### Solubilization of Powdered CDM for Hydrogel Synthesis.

1. Transfer CDM powder to glass vial that will be used for solubilization.
   a. The dECM is highly charged making it difficult to handle. Be very patient and move slowly. Incubating CDM powder at −20° C. before handling helps reduce the charge effects, and avoid grasping your palm around the container or it will stick to the sides of the vial.
   b. Use a metal spatula to handle the CDM powder between the two containers.
2. Determine mass of pepsin and volume of HCl needed. For every 100 mg of CDM, add 6 mg of pepsin and 10 mL of 0.01 HCl.
3. Add the pepsin/HCl solution to the.
4. Place the stir bar in the glass vial and place on the stir plate.
   i. Place the vial slightly off-centered on the stir plate to where the stir bar barely drags along the edge of the vial.
   ii. Turn the stir speed to medium-slow (˜200 rpm). The speed may need to be slightly increased during solubilization if viscosity slows it down.
   iii. Use tape to hold the vial in place.
5. Incubate for 12 hours while stirring at room temperature.
   a. The digest can desirably be viscous and should appear homogeneous.
   b. In some embodiments, the present Example employs 11 or 12 hours.
6. After solubilization, immediately proceed to hydrogel synthesis or freeze aliquots in −80° C.
   a. In subsequent steps, the resulting solubilized CDM is referred to as “CDM pre-gel solution.”

### Reagents for Hydrogel Synthesis

dECM digest
10× PBS
0.1 M NaOH

### Hydrogel Preparation

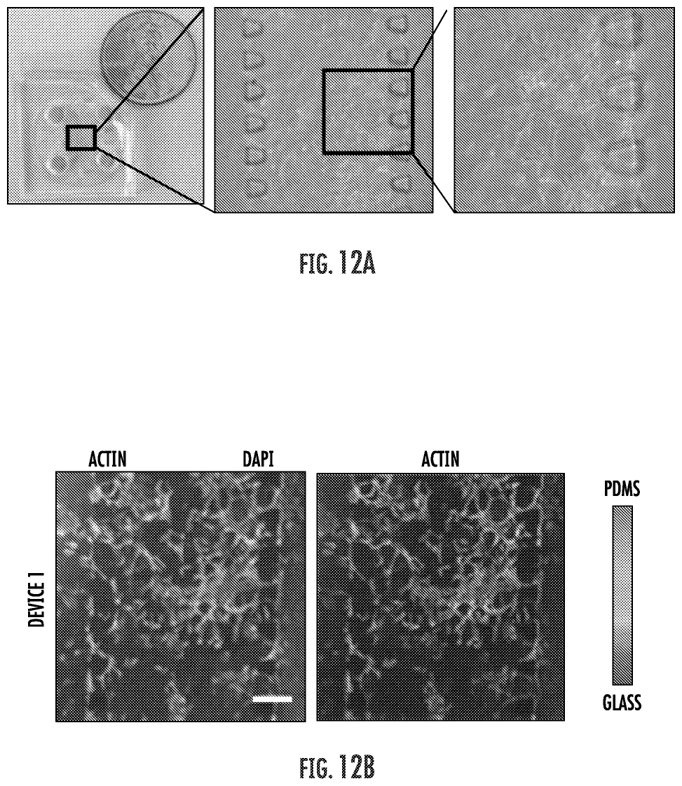

Hydrogel Preparation results in a viscous solution that contains digested CDM. This solution is then used to form the hydrogel that serves as the cell substrate.
1. If using frozen aliquot, thaw at room temperature.
2. Place all reagents on ice.
3. If supplementing with rat-tail collagen, determine the ratio of CDM pre-gel solution to collagen, and add collagen solution directly to the CDM pre-gel solution.
   i. For following calculations, add the necessary volume of collagen to the CDM pre-gel solution volume and use resulting total volume for CDM hydrogel preparation.
   ii. A CDM/collagen hydrogel could be used within a microfluidic device (FIG. 11).
4. If adding genipin, determine the final concentrations of genipin desired in the gel. Calculate the volume of genipin to add based on the concentration of the genipin stock (0.1105 M) and the volume of the pre-gel solution being used.
   i. A CDM-genipin hydrogel could be used within a microfluidic device.
5. Add 10× PBS to equilibrate isotonic balance.
   i. For 600 uL of CDM pre-gel solution, add 100 μL of 10× PBS.
6. Add 0.1 M NaOH to achieve a physiological pH of 7.5 in the total solution.
7. If completing turbidity assay, transfer pre-gel solution to well plate. If not, place the pre-gel solution in the incubator at 37° C. for 1 hr.

### Example 3

### Results

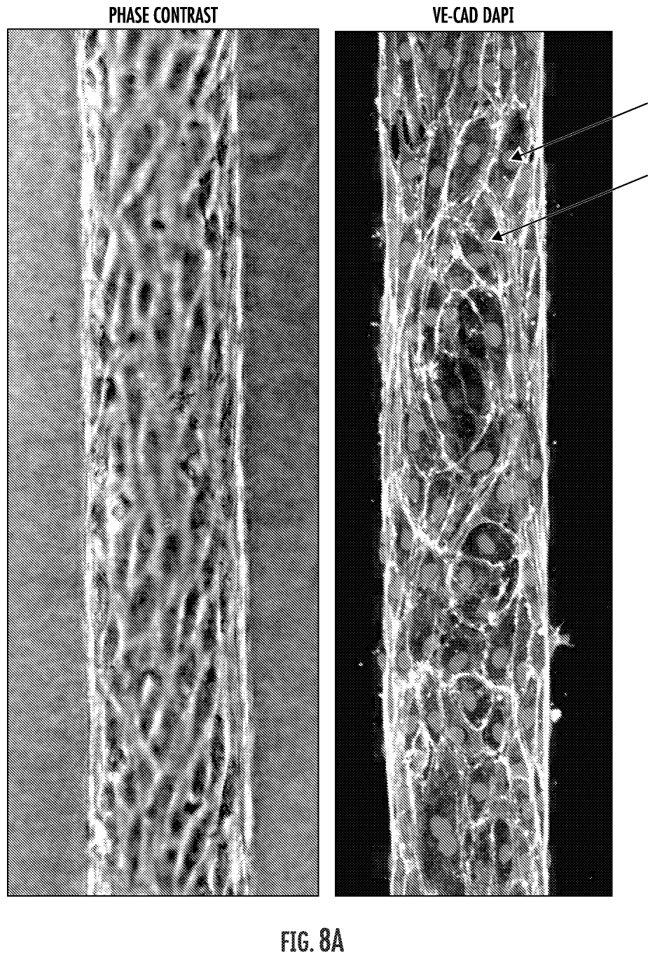

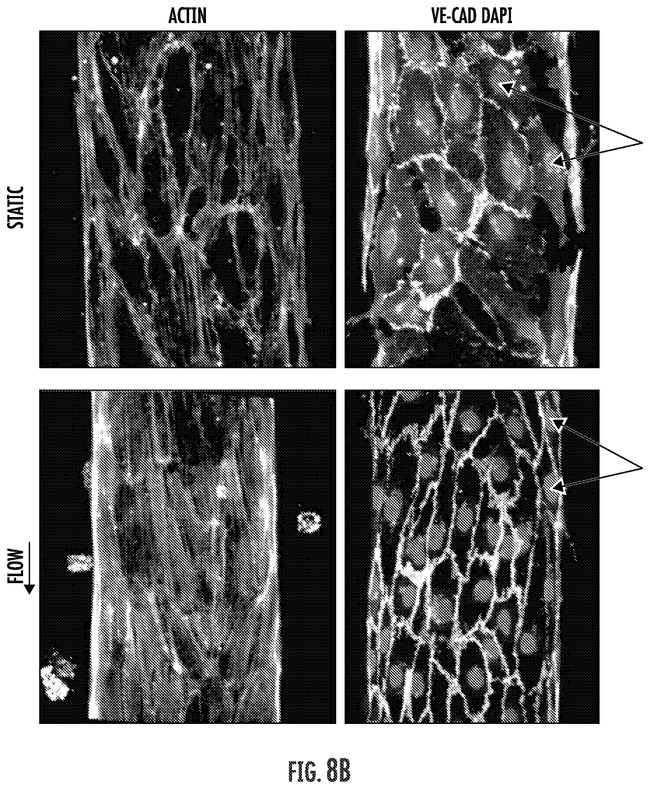

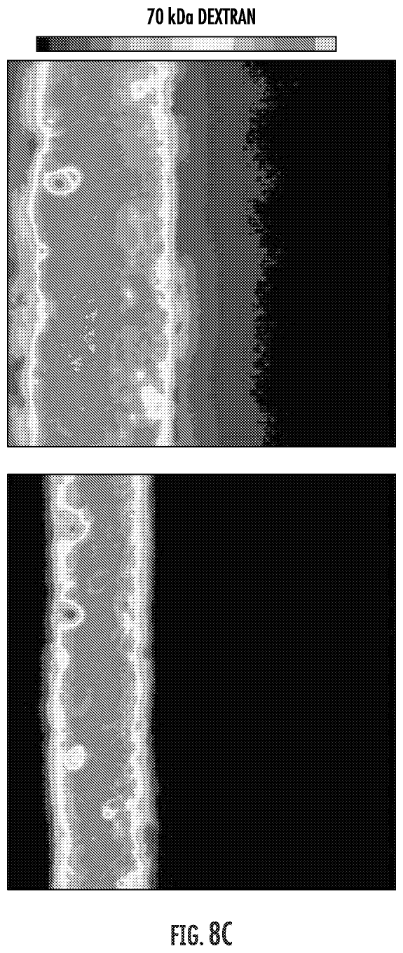

The microfluidic devices fabricated as discussed above can be used for effectively modeling a cellular transport barrier in a flow environment. As shown in FIG. 8A, for example, the present microfluidic devices can be used as a platform to create a human engineered microvessel. Connecting devices to a commercially available syringe pump to apply flow to impart a shear stress (5 dyne/cm²) (Bottom Row FIG. 8B) at the wall induces cell alignment and promotes adherens junction formation as compared with a static condition (Top Row FIG. 8B). Arrows in 8A and 8B show DAPI stained nuclei of microvessel. As shown in FIG. 8C, the intensity of Texas Red-conjugated 70-kDa dextran demonstrates that flow (bottom; flow applied via a syringe pump to induce 5 dyne/cm² wall shear stress) enhances the human engineered microvessel barrier function as compared with the static condition which allows for dextran to leak from the vessel into the surrounding biomaterial structure (top).

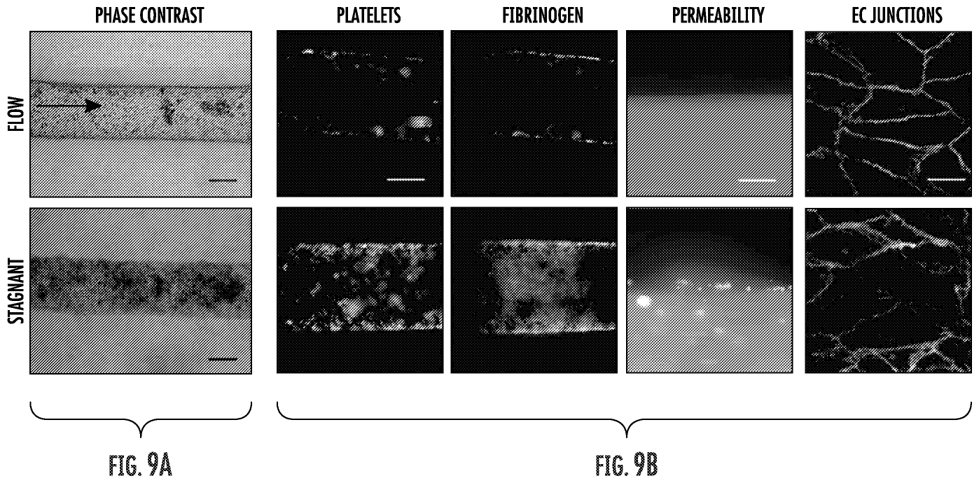

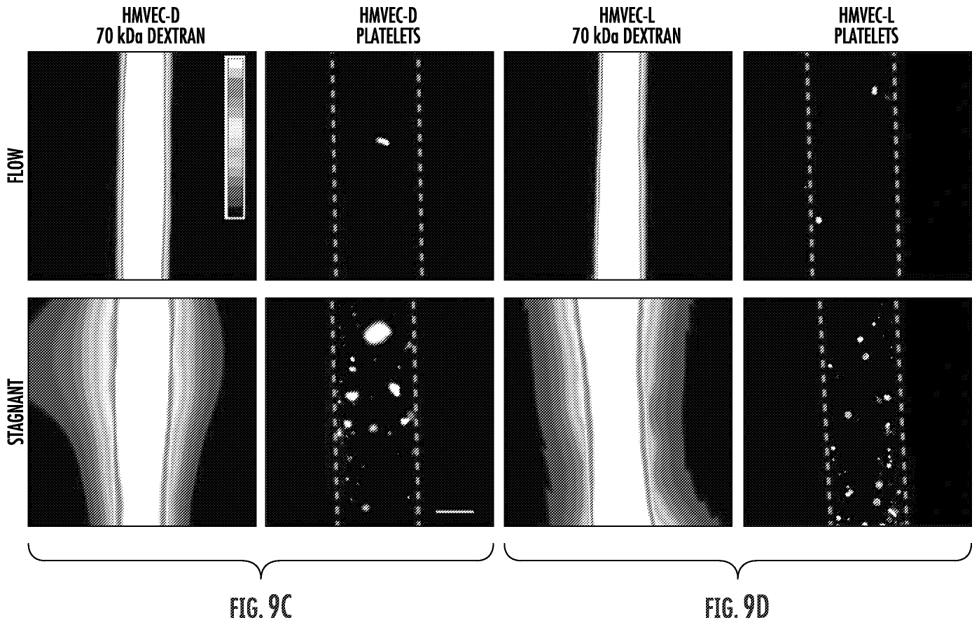

Further referring to FIGS. 9A and 9B, the microfluidic device can be used with patient blood and plasma. The Arrow in FIG. 9A designates the direction of the flow of the patient blood and plasma. FIG. 9A illustrates phase contrast images of the present microfluidic device seeded with primary human endothelial cells (hMVEC-D) after introduction of whole blood from patient finger-prick, with perfusion for about 5 min, and wash with PBS. Chips cultured under static conditions caused clotting as shown by accumulation of phase-dense cells and fibrin. As shown in FIG. 9B, chips perfused with platelet rich patient plasma from finger prick demonstrate the ability to quantify platelet adhesion, fibrin-rich clot formation, changes in vascular permability, and endothelial cell phenotype.

Further referring to FIGS. 9C and 9D, the microfluidic device can also be used with different primary adult human endothelial cells for patient or disease specific measurement of vascular health. As shown in FIG. 9C, vessels are formed with human adult dermal microvascular endothelial cells (hMVEC-D), and as shown in FIG. 9D, vessels are formed with human adult lung microvascular endothelial cells. While not wishing to be bound by theory, vessels may be formed from culture patient-derived primary cells. The top row of FIGS. 9C and 9D show platelets from a patient finger prick and the bottom row contains images of dextran permeable taken using flow versus static flow forces. Here, vascular health metrics can be developed based on dextran permeability or leakage (bottom row) and patient-specific adhesion (top row).

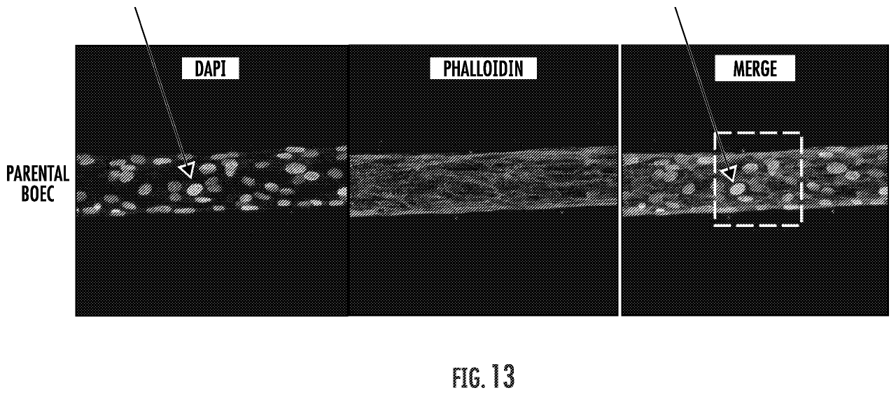

In some embodiments, culture patient-derived primary cells comprise cells derived from patient blood draws (FIG. 13). Referring to FIG. 13, arrows point to DAPI stained nuclei of endothelial cells. Phalloidin stains actin filaments of endothelial cells. Merger of these images (inside box FIG. 13) shows viable cells in vessel vasculature. Endothelial cells are from patient blood draws.

In some embodiments, the vessels comprise methods and compounds for patient-specific disease modeling. In some embodiments, the vessels comprise methods and compounds for drug screening.

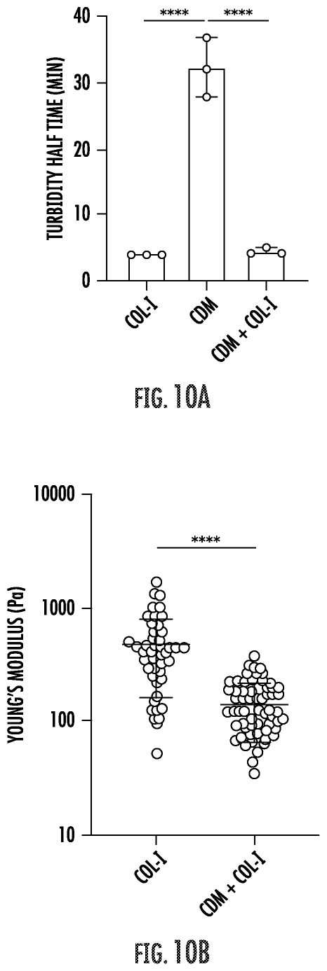

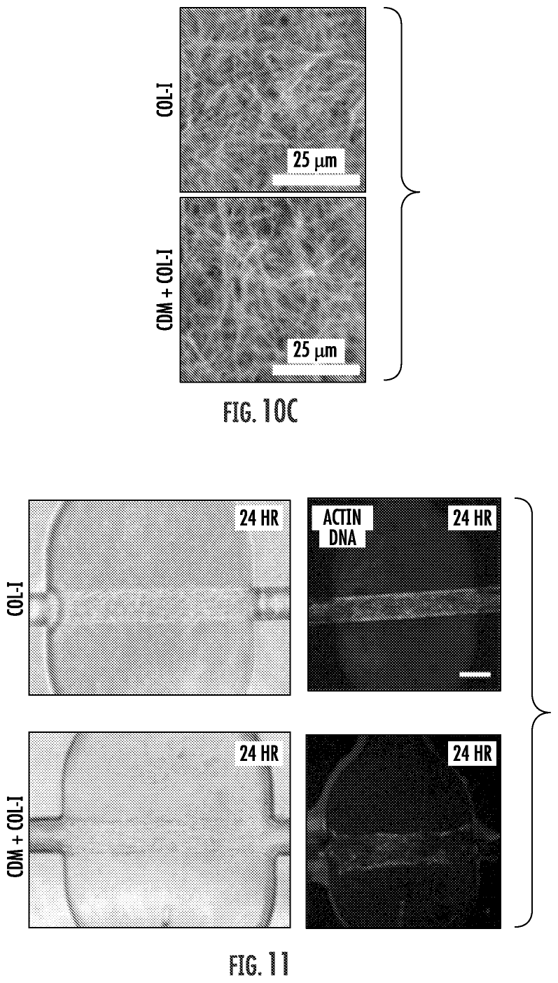

Further referring to FIG. 10A turbidity analysis demonstrates the formation of cell-derived matrix (CDM) hydrogels compared to rat-tail derived collagen type I (COL-1) and CDM supplemented with collagen type I (CDM+COL-1). As such, it has been demonstrated that the components to create these vessels come completely from patient-donor materials (i.e., cells, perfusate, blood and matrix). Referring to FIG. 10B, Young's modulus, as determined by nanoindentation, demonstrates the ability to form hydrogels with physiologic mechanical properties. This analysis demonstrates the ability to synthesize hydrogels from cell culture substrates. Referring to FIG. 10C, scanning electron microscopy images show fibril structure of hydrogels.

Further referring to FIG. 11, microfluidic devices can be patterned to contain blood vessels consisting of human umbilical vein endothelial cells embedded in cell-derived matrix hydrogels.

Further referring to FIG. 12A and 12B, human umbilical vein endothelial cells seeded within cell-derived matrix hydrogels spontaneously form interconnected microvascular networks. Boxes shown in FIG. 12A illustrate that these networks are being formed within microfluidic devices. In the right most image, the interconnected networks can be seen near the patterned trapezoidal shapes in the device. FIG. 12B shows vascular networks formed in the CDM hydrogels with the cells stained with phalloidin to show actin within the cells. In the black and white version, the left and right panels appear redundant. Color scale indicates depth to highlight that there is some distribution of these cells in the direction that is into and out a three dimensional space.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

1. A method for producing a microfluidic device, the method comprising:
producing a first housing portion and a second housing portion;
securing the second housing portion to the first housing portion;
enclosing a three-dimensional biomaterial structure between the first housing portion and the second housing portion; and
forming one or more channel within the biomaterial structure, the one or more channel being configured to model a hollow tissue structure;
wherein the biomaterial structure and the one or more channel are configured for modeling a cellular transport barrier in a flow environment.

2. The method of claim 1, wherein producing the first housing portion and the second housing portion comprises using a fabrication protocol selected from the group consisting of photolithography, injection molding, and embossing.

3. The method of claim 1, wherein one or both of the first housing portion or the second housing portion comprises a substantially optically transparent material.

4. The method of claim 1, wherein enclosing the three-dimensional biomaterial structure between the first housing portion and the second housing portion comprises positioning the first housing portion and the second housing portion to apply fluid pressure to the biomaterial structure.

5. The method of claim 1, wherein enclosing the three-dimensional biomaterial structure between the first housing portion and the second housing portion comprises:
inserting liquid biomaterial between the first housing portion and the second housing portion; and
polymerizing the liquid biomaterial to form the three-dimensional biomaterial structure.

6. The method of claim 1, wherein the biomaterial structure comprises a hydrogel.

7. The method of claim 1, wherein enclosing the three-dimensional biomaterial structure between the first housing portion and the second housing portion comprises:
positioning a lyophilized hydrogel between the first housing portion and the second housing portion; and
supplying water to the lyophilized hydrogel to reconstitute the biomaterial structure.

8. The method of claim 1, wherein the biomaterial structure comprises a human-cell-derived extracellular matrix.

9. The method of claim 1, wherein one or both of the first housing portion or the second housing portion comprises one or more alignment feature; and
wherein forming each of the one or more channel comprises:
aligning a tubular structure with the one or more alignment feature prior to enclosing the three-dimensional biomaterial structure between the first housing portion and the second housing portion; and
removing the tubular structure to create a cylindrical void that defines the one or more channel within the biomaterial structure.

10. The method of claim 9, wherein the tubular structure comprises a needle having a diameter in a range from about 0.12 mm to about 0.35 mm.

11. The method of claim 1, wherein the tubular structure is coated with a material configured to inhibit adhesion of the tubular structure to the biomaterial structure.

12. The method of claim 1, wherein the tubular structure comprises a dissolvable needle.

13. The method of claim 1, further comprises seeding cells in the biomaterial structure.

14. A microfluidic device comprising:
a first housing portion;
a second housing portion secured to the first housing portion;
a three-dimensional biomaterial structure enclosed between the first housing portion and the second housing portion; and
one or more channel formed within the biomaterial structure;
wherein the biomaterial structure and the one or more channel are configured for modeling a cellular transport barrier in a flow environment.

15. The device of claim 14, wherein one or both of the first housing portion or the second housing portion comprises a substantially optically transparent material.

16. The device of claim 14, wherein the first housing portion comprises one or more alignment features configured to facilitate positioning of the one or more channel in the biomaterial structure.

17. The device of claim 14, wherein the first housing portion and the second housing portion are configured to apply fluid pressure to the biomaterial structure.

18. The device of claim 14, wherein the biomaterial structure comprises a hydrogel.

19. The device of claim 14, wherein the biomaterial structure comprises a human-cell-derived extracellular matrix.

20. The device of claim 14, comprising one or more media port formed in the second housing portion in communication with the one or more channel.

21. The device of claim 14, comprising a flow system in communication with one of the one or more media port, wherein the flow system is configured to generate fluid flow through the one or more channel.

22. The device of claim 14, comprising one or more extracellular matrix port formed in the second housing portion in communication with the biomaterial structure.

23. The device of claim 14, comprising cells seeded in the biomaterial structure.

24. A method for producing a microfluidic device, the method comprising:
producing a device housing comprising one or more internal cavity enclosed therein;
positioning a three-dimensional biomaterial structure within the one or more cavity in the device housing, wherein the biomaterial structure comprises a human-cell-derived extracellular matrix; and
forming one or more channel within the biomaterial structure, the one or more channel being configured to model a hollow tissue structure;
wherein the biomaterial structure and the one or more channel are configured for modeling a cellular transport barrier in a flow environment.

25. The method of claim 24, wherein the device housing comprises a substantially optically transparent material.

26. The method of claim 24, wherein positioning the three-dimensional biomaterial structure within the one or more cavity in the device housing comprises:
inserting liquid biomaterial within the one or more cavity in the device housing; and
polymerizing the liquid biomaterial to form the three-dimensional biomaterial structure.

27. The method of claim 24, wherein the biomaterial structure comprises a hydrogel.

28. The method of claim 24, wherein positioning the three-dimensional biomaterial structure within the one or more cavity in the device housing comprises:
positioning a lyophilized hydrogel within the one or more cavity in the device housing; and
supplying water to the lyophilized hydrogel to reconstitute the biomaterial structure.

29. The method of claim 24, wherein forming each of the one or more channel comprises:
positioning a tubular structure within the one or more cavity in the device housing prior to enclosing the three-dimensional biomaterial structure within the one or more cavity in the device housing; and
removing the tubular structure to create a cylindrical void that defines the one or more channel within the biomaterial structure.

30. The method of claim 29, wherein the tubular structure comprises a needle having a diameter in a range from about 0.12 mm to about 0.35 mm.

31. The method of claim 24, wherein the tubular structure is coated with a material configured to inhibit adhesion of the tubular structure to the biomaterial structure.

32. The method of claim 24, wherein the tubular structure comprises a dissolvable needle.

33. The method of claim 24, further comprises seeding cells in the biomaterial structure

34. A microfluidic device comprising:
a device housing comprising one or more cavity enclosed therein;
a three-dimensional biomaterial structure positioned within the one or more cavity of the device housing, wherein the biomaterial structure comprises a human-cell-derived extracellular matrix; and
one or more channel formed within the biomaterial structure;
wherein the biomaterial structure and the one or more channel are configured for modeling a cellular transport barrier in a flow environment.

35. The device of claim 34, wherein the device housing comprises an optically transparent material.

36. The device of claim 34, wherein the biomaterial structure comprises a hydrogel.

37. The device of claim 34, comprising one or more media port formed in the device housing in communication with the one or more channel.

38. The device of claim 37, comprising a flow system in communication with one of the one or more media port, wherein the flow system is configured to generate fluid flow through the one or more channel.

39. The device of claim 34, comprising one or more extracellular matrix port formed in the second housing portion in communication with the biomaterial structure.

40. The device of claim 34, comprising cells seeded in the biomaterial structure.
